(19) United States
(12) Patent Application Publication
(10) Pub. No.: US 2006/0089660 A1
(43) Pub. Date: Apr. 27, 2006

(54) System and method for endoscopic treatment of tissue

(76) Inventors: Zahid A. Saeed, Cincinnati, OH (US); Nib Soehendra, Henstedt-Ulzburg (DE); John Mugan, Moycullen (IE)

Correspondence Address: BRINKS HOFER GILSON & LIONE/CHICAGO/COOK, PO BOX 10395, CHICAGO, IL, 60610, US

(21) Appl. No.: 11/127,554

(22) Filed: May 12, 2005

Related U.S. Application Data
(60) Provisional application No. 60/571,279, filed on May 13, 2004.

(51) Int. Cl. A61B 17/10

(52) U.S. Cl. 606/139

(57) ABSTRACT

A system and method for endoscopic treatment of tissue is disclosed. An endoscope has an operating channel. A mounting component has a first threading channel that fits into the operating channel of the endoscope. A ligating barrel fits onto a distal end of the endoscope. A medical instrument is inserted through the first threading channel, the operating channel, and the ligating barrel to treat tissue.

The application claims benefit of U.S. Provisional Application No. 60/571,279 filed May 13, 2004.

### TECHNICAL FIELD

This system is related to medical devices and more particularly, to endoscopic treatment of tissue.

### BACKGROUND OF THE INVENTION

The treatment of tissue encompasses a variety of techniques such as electrocauterization, heat therapy, resection (removal of tissue), and sclerotherapy (the injection of medicine into target tissue). These treatment techniques usually involve the passing of medical instruments through the operating channel of the endoscope. The endoscope permits minimally invasive access, as well as visualization and suction aids.

Another technique that frequently utilizes the operating channel of the endoscope is ligation, which involves applying a band or ligature around a vessel or portion of tissue, thereby cutting off blood or fluid flow and causing the tissue to necrose and separate from adjacent healthy tissue. Ligation is widely used to treat a number of medical tissue conditions, including, but not limited to, hemorrhoids, polyps, ballooning varices, and other types of lesions, including those that are cancerous. Typically, ligators are also used with a suction or vacuum means to draw the tissue into the distal tip, whereby the band is deployed over the base of the diseased tissue to cut off blood flow. The ligating device is typically activated by retracting a line (string, wire, or cable) that is attached to the ligator at the distal end of an endoscope and is threaded through the operating channel of the endoscope to the proximal end of the instrument. The ligator can be activated by mechanically pulling the activating line by means of a hand-operated reel or trigger, or a motor drive mechanism. Various other ligating devices use cooperating inner and outer members that slide the individual bands by pushing or pulling them from the tip of the inner or outer member, the bands being preloaded onto the inner or outer member prior to deployment.

To prevent having to withdraw the instrument from the patient, reload, and reintroduce it for treating additional tissue or vessels, devices have been developed capable of sequentially delivering multiple bands that are preloaded, thus shortening the procedure time and improving patient comfort. Multiple band ligating devices include designs that individually tether or otherwise secure the bands to the dispenser and then release them sequentially as needed, often by use of one or more strings extending to the proximal end.

It is often desirable to combine another endoscopic procedure, such as sclerotherapy or tissue removal with a surgical snare, with ligation. However, while the operating channel of the endoscope is often large enough to accommodate more than just an activating line from a ligator, combining the medical instruments necessary for the second procedure with the ligator can be problematic. Thus, there is a need for a ligating device that can be combined with other medical instruments in endoscopic procedures.

### BRIEF SUMMARY

The present invention provides a system and method for endoscopic treatment of tissue. In particular, a system is provided with an endoscope, a mounting component, a ligating barrel and a medical instrument. The endoscope has an operating channel. The mounting component has a first threading channel that fits into the working channel of the endoscope. The mounting component has a diameter of at least 3.3 millimeters. The ligating barrel fits onto a distal end of the endoscope. The medical instrument is inserted through the first threading channel, the operating channel, and the ligating barrel to treat tissue.

These and other features of the invention will become apparent upon review of the following detailed description of the presently preferred embodiments of the invention, taken into conjunction with the appended figures.

### BRIEF DESCRIPTION OF THE DRAWINGS

FIG. 1 is a longitudinal fragmented view of an endoscope with a ligating system.

FIG. 2 is a top plan view of the activating mechanism of the ligating system of FIG. 1.

FIGS. 3A-3C are views of a mounting component of the activating mechanism of FIG. 2.

FIGS. 4A-4E are views of a base of the activating mechanism of FIG. 2.

FIG. 5 is a view of a spool of the activating mechanism of FIG. 2.

FIGS. 6-7 are views of the activating mechanism of FIG. 2 with the incorporation of a surgical snaring system in the present invention.

FIGS. 8A-8B are views of a disengaged mode of operation of the activating mechanism of FIG. 2 wherein knob 60 is free to rotate in either direction (A or B).

FIGS. 9A-9B are views of an engaged mode of operation of the activating mechanism of FIG. 2 wherein knob 60 is only free to rotate in a single direction (A).

FIG. 10 is a view of the activating mechanism of FIG. 2 prior to insertion into the operating channel of the endoscope of FIG. 1.

FIG. 11 is a view of the activating mechanism of FIG. 2 after insertion into the operating channel of the endoscope of FIG. 1.

FIG. 12 is a cross-sectional view of a ligating barrel with the incorporation of the surgical snaring system in another embodiment of the present invention.

### DETAILED DESCRIPTION OF THE DRAWINGS AND THE PRESENTLY PREFERRED EMBODIMENTS

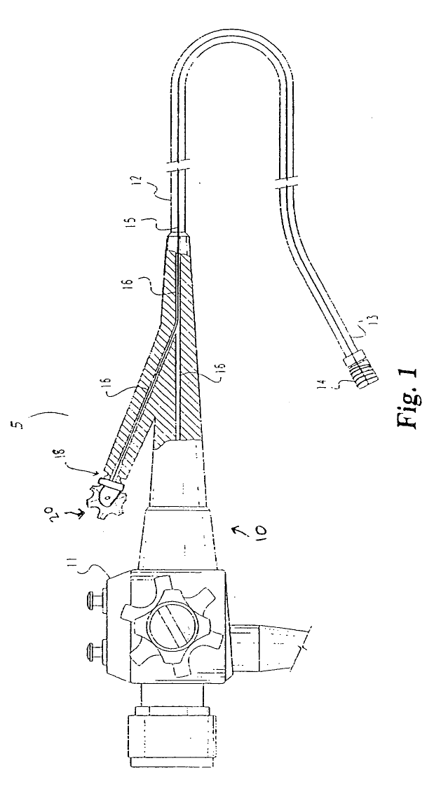

Referring now to the drawings, there is shown in FIG. 1 a ligating system 5 with endoscope 10. Ligating system 5 has a ligating barrel 14 attached to the distal end of endoscope 10. Ligating barrel 14 is generally shown in FIG. 1. A more detailed description of barrel 14 is shown in U.S. Pat. No. 5,624,453, which disclosure is hereby incorporated by reference. Endoscope 10 is a conventional endoscope with an operating control portion 11, a flexible section 12, and a distal end portion 13.

Ligating barrel 14 is located at distal end portion 13 of endoscope 10 and includes an activation line 15. Endoscope 10 also includes operating channel 16 which extends through endoscope 10 from ligating barrel 14 to both operating control portion 11 and to proximal opening 18. Activation line 15 is threaded from ligating barrel 14 through operating channel 16 and exits through proximal opening 18. Barrel 14 is preferably of a hard plastic clear polycarbonate for maximum durability and visibility.

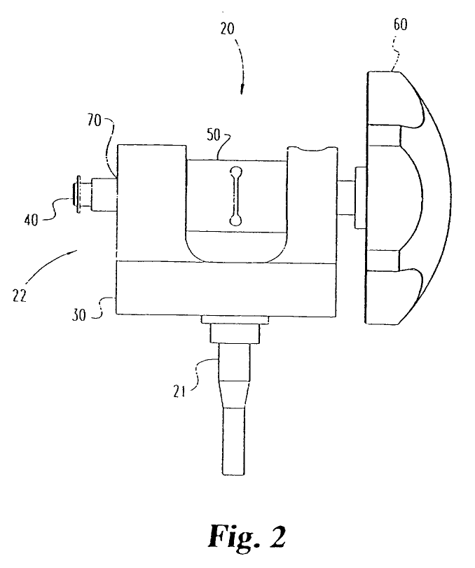

FIG. 2 is a top plan view of an activating mechanism 20 for ligating system 5. Activating mechanism 20 includes mounting component 21 and activating component 22. Activating component 22 includes base 30, drive pin 40, spool 50, knob 60 and roller clutch 70. Mounting component 21 attaches to activating component 22 and is used to mount activating mechanism 20 to endoscope 10 at proximal opening 18.

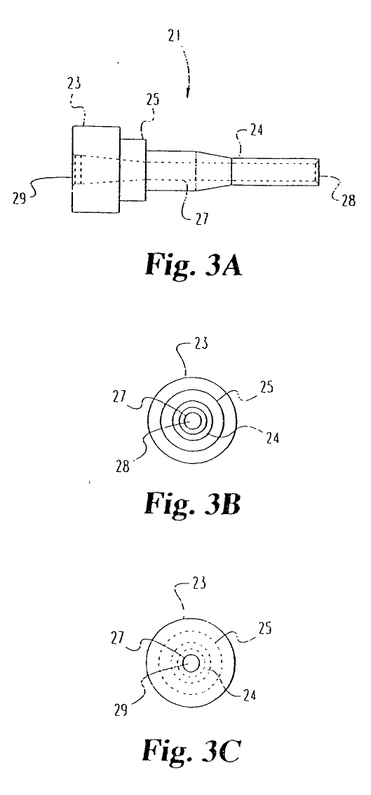

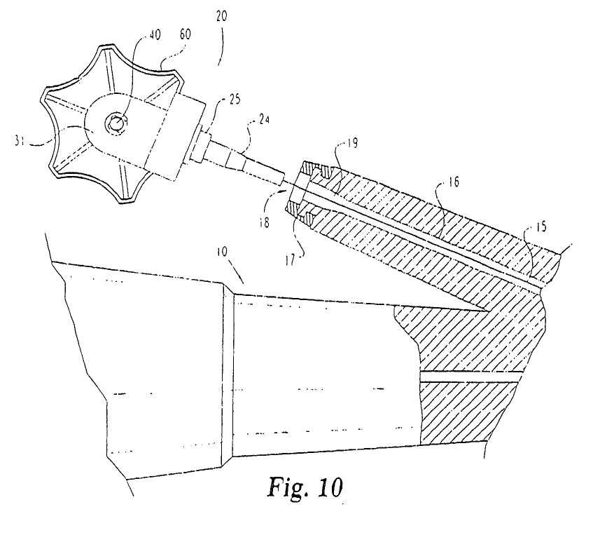

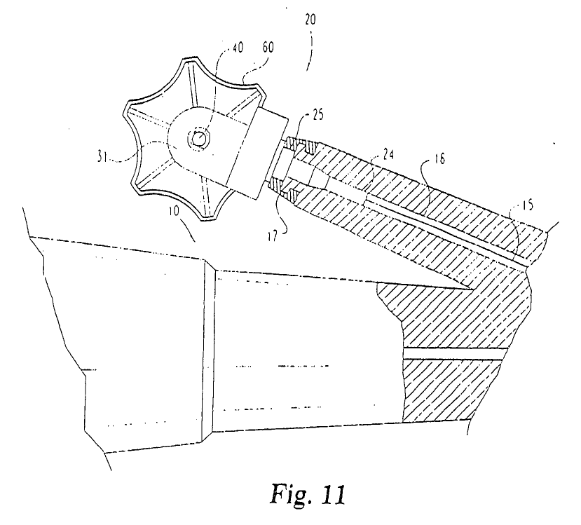

FIG. 3A is a view of mounting component 21. Mounting component 21, which is preferably formed of resilient material such as a plastic or a polyurethane, and more preferably formed of a strong material such as aluminum, includes coupling portion 23, friction-fit portion 24, and outer sealing portion 25. Coupling portion 23 is adapted to fixedly attach to activating component 22. Upon insertion into operating channel 16 through proximal opening 18, friction-fit portion 24 is sized to form a friction fit within operating channel 16. Where endoscope 10 is provided with a sealing member 17 (as shown in FIGS. 10 and 11), outer sealing portion 25 is adapted to seat in and seal with sealing member 17.

Mounting component 21 also provides first threading channel 27 which extends through mounting component 21. First opening 28 of first threading channel 27 is shown in FIG. 3B and second opening 29 of first threading channel 27 is shown in FIG. 3C. After mounting component 21 has been inserted into operating channel 16, activation line 15 can be threaded into first opening 28 and out of second opening 29 for connection to activating component 22.

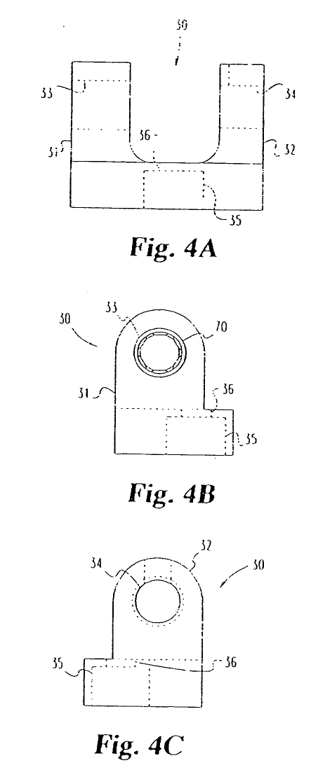

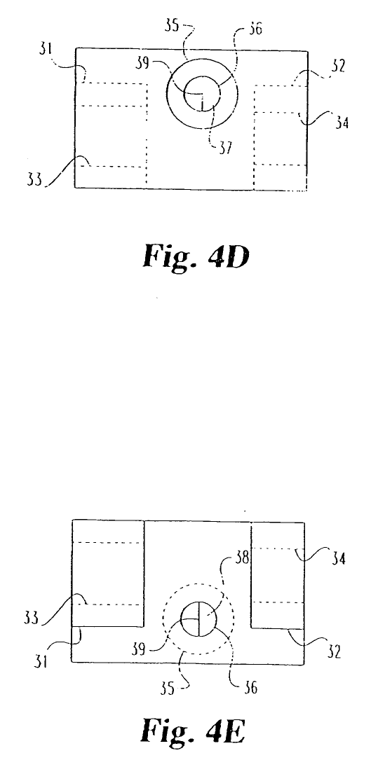

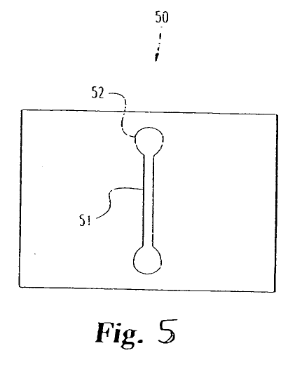

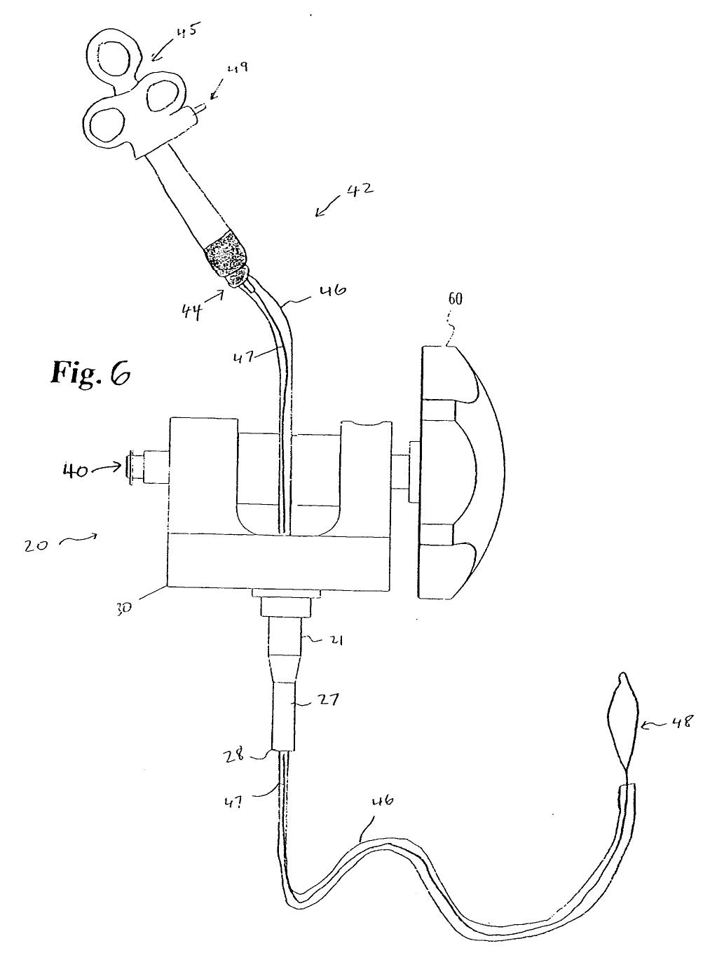

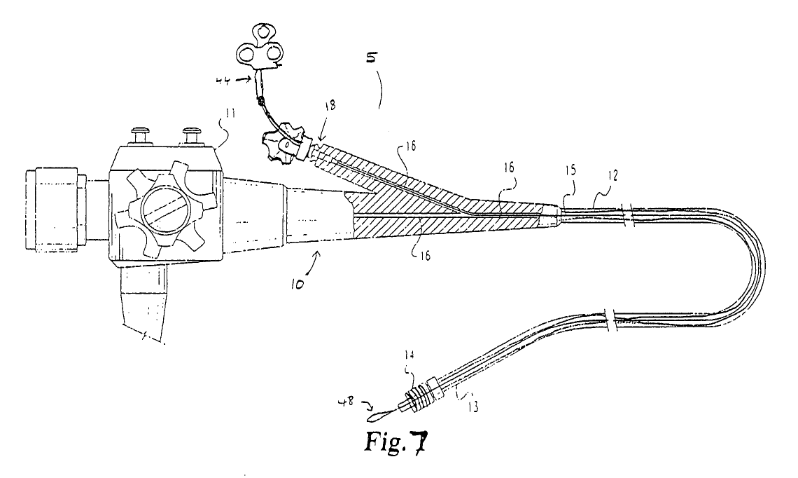

FIG. 4A is a frontal view of base 30. Base 30 includes first arm 31 and second arm 32. As shown in FIG. 4B, first arm 31 provides first receiving opening 33 and as shown in FIG. 4C, second arm 32 provides second receiving opening 34. Base 30 further includes recess 35 located on the bottom side portion of base 30 and second threading channel 36 which extends through recess 35 to the top side of base 30. FIG. 4D is a view of the bottom side of base 30, and further shows recess 35 and first opening 37 of second threading channel 36. FIG. 4E is a view of the top side of base 30, showing the top side of first arm 31 and second arm 32, and second opening 38 of second threading channel 36 with inner sealing member 39 located therein. When activation line 15 is threaded through mounting component 21, it is further threaded into first opening 37, through inner sealing member 39, and out of second opening 38 of base portion 30.

FIG. 5 is a side view of spool 50. Hole 52 and slot 51 receive activation line 15 from second opening 38 of base 30. Activation line 15 is coupled to hole 52 and slot 51 by slipping activation line 15 into slot 51 and placing a knot in activation line 15 into hole 52. Drive pin 40 engages spool 50, permitting spool 50 to rotate in the same direction as drive pin 40 when the drive pin 40 is rotated by knob 60. The connection also prevents drive pin 40 from dislodging out of second receiving opening 34.

FIGS. 6-7 illustrate the combination of activating mechanism 20 with the incorporation of a surgical snaring system 42. Surgical snaring system 42 includes snare 44, handle 45, catheter 46, flexible wire 47, operating loop 48 and electrical connector 49. Catheter 46 extends through openings 37 and 38 of second threading channel 36 (shown in FIGS. 4A-4E) of base 30. Catheter 46 also extends through openings 28 and 29 of first threading channel 27 of mounting component 21. Catheter 46, flexible wire 47 and operating loop 48 then extend through operating channel 16 of endoscope 10 and can extend through ligating barrel 14.

To facilitate the extension of activation line 15 and catheter 46 all the way through first threading channel 27, first threading channel 27 and first opening 28 can be enlarged via boring from a typical diameter of about 2-3 mm to a new diameter of about 3.3-5 mm. A 2-3 mm diameter threading channel and first opening allows the physician to use a snare sized at about 5 FR. A higher diameter threading channel and first opening (3.3-5 mm) permits use of a larger sized snare, as much as 7 FR. Regardless of the size of the threading channel and first opening, it is imperative that outer sealing portion 25 forms a seal with sealing member 17 so that there is no pressure loss in the vacuum needed during endoscopic procedures.

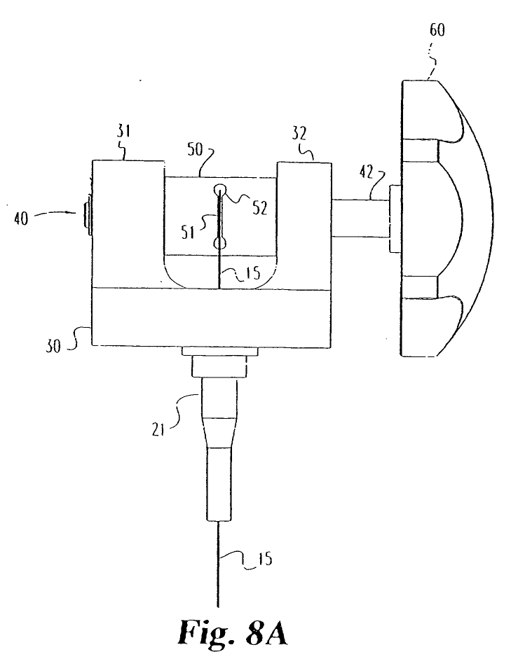

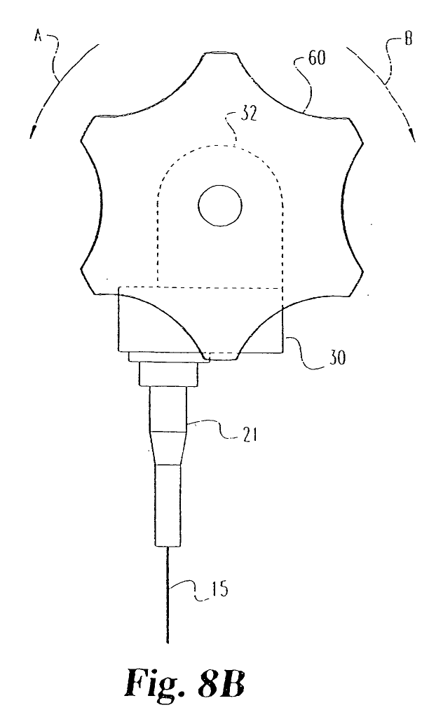

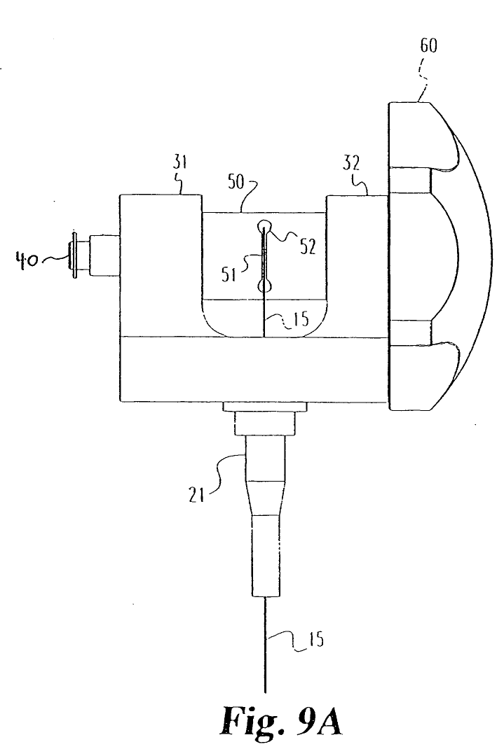

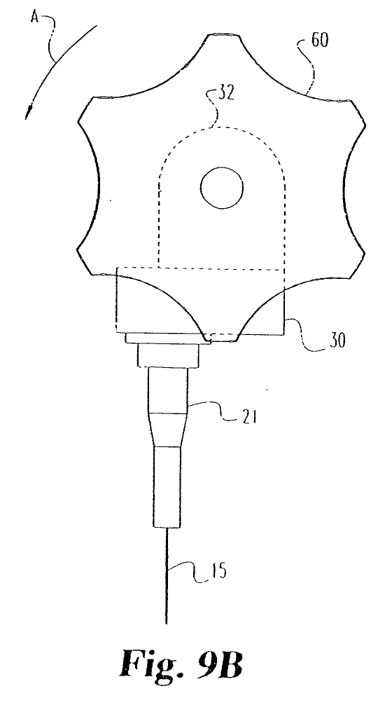

FIGS. 8A, 8B, 9A and 9B are views of two modes of operation for activating mechanism 20. FIG. 8A depicts the disengaged mode of operation. Activation line 15 and catheter 46 are threaded through mounting component 21 and base 30. Activation line 15 is coupled to spool 50 and ligating bands. The use of an activation line or lines to release one or more ligating bands is well known in the art and is described in U.S. Pat. No. 6,730,101, the entire contents of which are hereby incorporated herein by reference. The disengaged mode of operation is based on a disengaged position of drive pin 40 within roller clutch 70. FIG. 8A discloses drive pin 40 being substantially disposed outside of second arm 32. FIG. 8A further shows drive pin 40 being substantially disposed within first arm 31. This is the disengaged position of drive pin 40. As shown in FIG. 8B, when drive pin 40 is in the disengaged position, knob 60 can be rotated freely in either direction (A or B). After the attachment of activation line 15 to spool 50, knob 60 can be turned in either direction to wrap activation line 15 around spool 50. To unwrap activation line 15, knob 60 can be rotated in the opposite direction of the first rotation.

FIG. 9A depicts the working mode of operation. The working mode of operation is also based on a working position of drive pin 40 within roller clutch 70. FIG. 9A discloses drive pin 40 as being substantially disposed within second arm 32. FIG. 9A further shows drive pin 40 as being substantially disposed outside of first arm 31 with drive pin 40 engaging contact with roller clutch 70. As shown in FIG. 9B, when drive pin 40 is in the engaged position, knob 60 can only be rotated in one direction. After the attachment of activation line 15 to spool 50, knob 60 can be rotated in one direction to wrap activation line 15 around spool 50 under controlled tension to release one or more ligating bands from ligating barrel 14 as desired. Upon completion of the ligation procedure, activating mechanism 20 may be reset in the disengaged mode to release the tension from activation line 15 and allow activation line 15 to be unwound from spool 50 and disconnected therefrom.

FIGS. 10 and 11 show how activating mechanism 20 is mounted to endoscope 10 by inserting mounting element 21 into operating channel 16. For this illustration, endoscope 10 is shown provided with sealing member 17 which is coupled to proximal opening 18 of endoscope 10. FIG. 10 shows the alignment of activating mechanism 20 with proximal opening 18 prior to insertion of mounting element 21 therein. FIG. 11 shows mounting element 21 inserted into operating channel 16. Upon insertion, friction-fitting portion 24 friction fits with receiving portion 19 of operating channel 16, and sealing portion 25 seals with sealing member 17 of endoscope 10. And as described earlier, activation line 15 is threaded into and attached to activating mechanism 20.

Accordingly, ligating system 5 can be used in conjunction with endoscope 10 by a physician for the treatment of tissue. First, the physician positions the ligating barrel 14 (and thus the distal end of the endoscope) over the target tissue. The physician then applies suction via the endoscope to a target tissue (or pseudo-polyp) and deploys one or more ligating bands around the pseudo-polyp. Once the pseudo-polyp has been banded, the physician may release the suction and insert snaring system 42 (with a snare as large as 7 FR if the threading channel and first opening is at least 3.3 mm in diameter) through threading channel 27, operating channel 16 and ligating barrel 14. The physician can position operating loop 48 to ensnare the pseudo-polyp above the ligating band(s) with the snaring system. Next, the physician can cut the pseudo-polyp through electric cautery via electrical connector 49 and then remove the pseudo-polyp with the operating loop. In particular, the present invention permits the physician to perform multiple band ligation and Endoscopic Mucosal Resection (EMR) in the esophagus and other portions of the gastrointestinal tract. This procedure can be repeated several times during one surgical session, increasing the physician's efficiency.

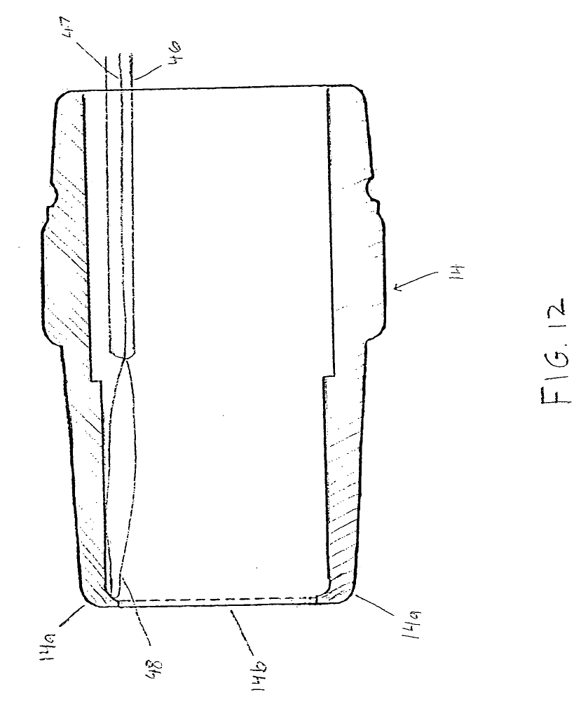

Alternatively, the physician can insert snaring system 42 through threading channel 27 and operating channel 16 at the beginning of the procedure, before applying suction and banding the pseudo-polyp. In this method, the physician can guide operating loop 48 all the way through operating channel 16 into ligating barrel 14. As illustrated in FIG. 12, the physician can then push operating loop 48 up against the distal rim 14a of the ligating barrel, just outside of the distal opening 14b of the barrel. This orientation serves to minimize the degree to which elements of the snaring system may interfere with the suction and banding steps by the physician. In order to further minimize this potential interference, a smaller sized snare (5 FR) can be used, which also permits the use of a smaller diameter (about 2 mm) for the threading channel and first opening.

Moreover, additional endoscopic devices and procedures can be combined with the activating mechanism of the ligating system due to the increased diameter in threading channel 27 and first opening 28. Thus, an injection needle for sclerotherapy or an endoscopic ultrasound (EUS) needle could be extended through threading channel 27 and first opening 28 into and through the operating channel 16 of endoscope 10. In addition, biopsy forceps could also be utilized (instead of suction) by extending the forceps through the threading channel to capture the tissue and withdraw the tissue into the ligating barrel before deploying ligating bands. These other medical instruments can be utilized to treat the target tissue either before or after the banding of the tissue. Furthermore, these instruments can be used serially in one surgical session. For example, a solution can first be injected into the submucosal layer of tissue to elevate the target tissue and separate it from the muscular layer before banding the target tissue with a ligating system and removing it with a snaring system. It should be noted that the present invention can also be used to treat vessels as well as tissue.

It is therefore intended that the foregoing detailed description be regarded as illustrative rather than limiting, and that it be understood that it is the following claims, including all equivalents, that are intended to define the spirit and scope of this invention.

1. A system for endoscopic treatment of tissue, comprising:
an endoscope having an operating channel, a proximal end and a distal end;
a mounting component with a first threading channel adapted to fit into the operating channel of the endoscope, the first threading channel having a diameter of at least 3.3 millimeters;
a ligating barrel adapted to fit onto the distal end of the endoscope; and
a medical instrument, wherein the medical instrument is inserted through the first threading channel, the operating channel, and the ligating barrel to treat target tissue.

2. The system of claim 1 further comprising one or more ligating bands on the ligating barrel and an activating component attached to the mounting component, wherein operation of the activating component releases one or more ligating bands from the ligating barrel.

3. The system of claim 2 further comprising an activation line coupled between the activating component and one or more ligating bands.

4. The system of claim 1 wherein the mounting component further comprises a coupling portion, a friction-fit portion, and an outer sealing portion.

5. The system of claim 2 wherein the activating component comprises a base, a drive pin, a spool, a knob, and a roller clutch.

6. The system of claim 1 wherein the medical instrument is a snare.

7. The system of claim 1 wherein the medical instrument is a needle.

8. The system of claim 1 wherein the medical instrument is a biopsy forceps.

9. The system of claim 7 wherein the needle is a sclerotherapy needle.

10. The system of claim 7 wherein the needle is an endoscopic ultrasound needle.

11. The system of claim 1 wherein the mounting component is formed of plastic or polyurethane.

12. The system of claim 1 wherein the mounting component is formed of aluminum.

13. A method for endoscopic treatment of tissue, comprising:
positioning a ligating barrel over target tissue, wherein the ligating barrel carries one or more ligating bands and is attached to a distal end of an endoscope, the endoscope having an operating channel into which a mounting component is adapted to fit;
drawing tissue into the ligating barrel;
deploying one or more ligating bands around the target tissue;
releasing the target tissue from the ligating barrel; and
inserting a medical instrument through the mounting component, the operating channel, and the ligating barrel to treat the target tissue.

14. The method of claim 13 wherein the tissue is drawn into the ligating barrel with suction.

15. The method of claim 13 wherein the one or more ligating bands are deployed with an activation line and an activating component, the activating component being attached to the mounting component, wherein operation of the activating component pulls the activation line proximally to release one or more ligating bands from the ligating barrel.

16. The method of claim 13 wherein the medical instrument is a snare.

17. The method of claim 13 wherein the medical instrument is a needle.

18. The method of claim 13 wherein the medical instrument is a biopsy forcep.

19. The method of claim 17 wherein the needle is a sclerotherapy needle.

20. The method of claim 17 wherein the needle is an endoscopic ultrasound needle.
